(19) United States
(12) Patent Application Publication
(10) Pub. No.: US 2025/0361156 A1
(43) Pub. Date: Nov. 27, 2025

(54) METHODS AND SYSTEMS FOR REAL-TIME WATER QUALITY ASSESSMENT

(71) Applicant: SAUDI ARABIAN OIL COMPANY, Dhahran (SA)

(72) Inventors: Ammal Fannoush Alanazi, Dammam (SA); James Ohioma Arukhe, Dhahran (SA)

(73) Assignee: SAUDI ARABIAN OIL COMPANY, Dhahran (SA)

(21) Appl. No.: 18/670,063

(22) Filed: May 21, 2024

(51) Int. Cl. C02F 1/00; C02F 1/52; C02F 1/68; G01N 15/00; G01N 15/02; G01N 33/18

(52) U.S. Cl. CPC C02F 1/008; C02F 1/5209; C02F 1/68; G01N 15/02; G01N 33/18; C02F 2209/02; C02F 2209/06; C02F 2209/105; C02F 2209/11; G01N 2015/0053

(57) ABSTRACT

Methods and systems for water quality assessment are disclosed. The method includes obtaining a first input data indicative of properties of a first liquid sample, the first input data including turbidity data and total suspended solids data for the first liquid sample, where the first liquid sample is acquired from a liquid source. The method further includes determining, using a computer processor and a machine learning model, a first predicted particle-size distribution of the first liquid sample based on the first input data, where particle-size distribution is controlled, at least in part, by a set of dosage parameters configurable by a water quality system. The method further includes determining, with an optimizer applied to the machine learning model, an optimal set of dosage parameters based on the first predicted particle-size distribution and adjusting the set of dosage parameters of the water quality system to the optimal set of dosage parameters.

### BACKGROUND

Traditional methods for monitoring water quality, particularly particle-size, involve manual sampling followed by laboratory analysis. This process is time-consuming, labor-intensive, and often leads to delays in responding to changes in water quality. Moreover, manual sampling may be prone to inconsistencies and does not provide real-time data, which is crucial for immediate decision-making in water treatment facilities. On the other hand, conventional tools such as turbidity meters and particle counters do not provide detailed information about particle-sizes. For example, while turbidity meters provide turbidity and total suspended solids (TSS) data, these meters do not output particle-size distribution. Similarly, particle counters do not effectively correlate turbidity and TSS data with particle-sizes and are typically expensive tools. Accordingly, there exists a need to determine the particle-size distribution in a water sample in real-time.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Embodiments disclosed herein generally relate to a method. The method includes obtaining a first input data indicative of properties of a first liquid sample, the first input data including turbidity data and total suspended solids data for the first liquid sample, where the first liquid sample is acquired from a liquid source. The method further includes determining, using a computer processor and a machine learning model, a first predicted particle-size distribution of the first liquid sample based on the first input data, where particle-size distribution is controlled, at least in part, by a set of dosage parameters configurable by a water quality system. The method further includes determining, with an optimizer applied to the machine learning model, an optimal set of dosage parameters based on the first predicted particle-size distribution. The method further includes adjusting the set of dosage parameters of the water quality system to the optimal set of dosage parameters.

Embodiments disclosed herein generally relate to a water quality system. The water quality system includes a plurality of sensors configured to measure property data of, at least, a liquid sample acquired from a liquid source. The water quality system further includes a control system configured to adjust a set of dosage parameters of one or more chemicals used by the water quality system. The control system is in communication with the plurality of sensors. The control system includes a processor and a memory storing instructions. The instructions, when executed by the processor, cause the processor to obtain input data for a first liquid sample from the plurality of sensors, the input data including turbidity data and total suspended solids data for the first liquid sample, where the first liquid sample is acquired from the liquid source. The instructions, when executed by the processor, further cause the processor to determine, using a machine learning model, a first predicted particle-size distribution of the first liquid sample based on the input data, where particle-size distribution is controlled, at least in part, by the set of dosage parameters. The instructions, when executed by the processor, further cause the processor to determine, with an optimizer applied to the machine learning model, an optimal set of dosage parameters based on the first predicted particle-size distribution. The instructions, when executed by the processor, further cause the processor to adjust the set of dosage parameters to the optimal set of dosage parameters.

Embodiments disclosed herein generally relate to a non-transitory computer readable medium storing instructions executable by a computer processor. The instructions, when executed by the processor, cause the processor to perform a method. The method includes obtaining input data indicative of properties of a first liquid sample, the input data including turbidity data and total suspended solids data for the first liquid sample, where the first liquid sample is acquired from a liquid source. The method further includes determining, using a machine learning model, a first predicted particle-size distribution of the first liquid sample based on the input data, where particle-size distribution is controlled, at least in part, by a set of dosage parameters configurable by a water quality system. The method further includes determining, with an optimizer applied to the machine learning model, an optimal set of dosage parameters based on the first predicted particle-size distribution. The method further includes adjusting the set of dosage parameters of the water quality system to the optimal set of dosage parameters.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 depicts a water treatment plant in accordance with one or more embodiments.

FIG. 2 depicts a system in accordance with one or more embodiments.

FIG. 3 depicts a flowchart in accordance with one or more embodiments.

FIG. 4 depicts a system in accordance with one or more embodiments.

FIG. 5 depicts a system in accordance with one or more embodiments.

FIG. 6 depicts a neural network in accordance with one or more embodiments.

FIG. 7A depicts a comparison of actual, predicted, and traditionally measured particle sizes for various samples.

FIG. 7B depicts a table comparing various metrics before and after implementation of a machine learning model as described herein.

FIG. 7C depicts a table demonstrating example maintenance and operation data in accordance with one or more embodiments.

FIG. 8 depicts a flowchart in accordance with one or more embodiments.

FIG. 9 depicts a computing system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. For example, a “flocculant” may include any number of “flocculants” without limitation.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

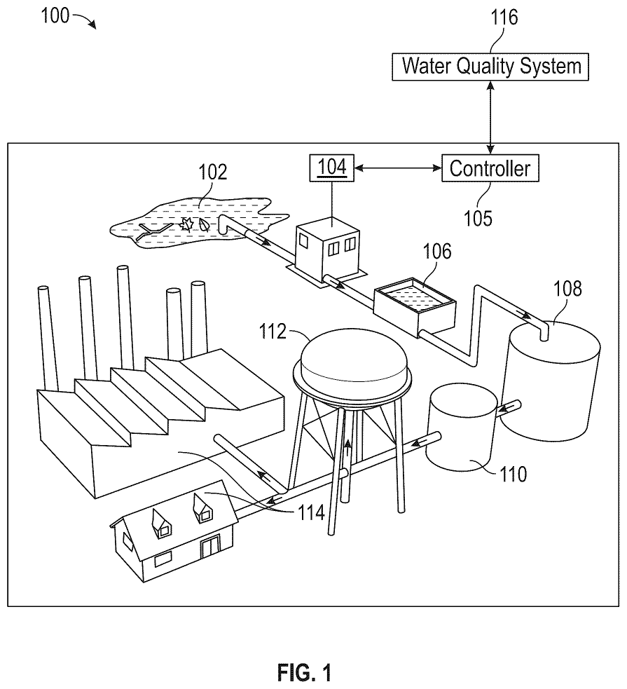

In the following description of FIGS. 1-9, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

Industrial water treatment processes can be of physical, chemical, or biochemical nature. Physical treatment processes include, for example, filtration, separation, and ion-exchange. Chemical treatment processes include flocculation, coagulation, and neutralization, among others. A water treatment plant can treat, or apply a treatment, to water. Determination of a treatment process implanted by the water treatment plant can depend on the toxicity of the compounds present in the water and sustainability regulations. Further, a proper determination of the particle-size distribution of a water sample is important to assess the effectiveness of a treatment process in a water treatment plant. For example, a filtration unit is designed to remove particles of specific sizes, and the particle-size distribution may be used to determine the effectiveness of the filtration processes. In such case, if the particle-size distribution is not within an expected range, the filtration efficiency may be reduced, and the water quality may be compromised. Traditionally, methods to determine particle-size involve manual sampling and laboratory analysis. A major disadvantage of these traditional methods is that they are often time-consuming, labor-intensive, and do not provide real-time data, which is crucial for immediate decision-making in water treatment plants. Further, conventional tools used to determine water quality (such as turbidity meters and automated particle counters) do not provide the particle-size distribution of a water sample. For example, while turbidity meters can give an indication of the overall level of total suspended solids (TSS), they do not provide detailed information about the size distribution of those particles. Taken together, these methods have their own set of potential errors and limitations and are unable to estimate the true particle-size distribution.

Embodiments disclosed herein generally relate to a water quality system that employs a machine learning model to determine the particle-size distribution of a water sample. The machine learning model is described in greater detail later in the instant disclosure. However, for now it is sufficient to state that the machine learning model determines the particle-size distribution of a water sample based on turbidity data and TSS data. Further, as will be described, the water quality system and the machine learning model are used for the optimization of the water treatment processes. The particle-size distribution can be determined using the machine learning model in real time. For example, in one or more embodiments, using the particle-size distribution as determined by the machine learning model, the water quality system model can enable a dynamic adjustment to the treatment process, such that particle aggregation is maximized. In such an embodiment, the dosage of the chemicals introduced by a chemical addition unit may be adjusted, in real-time or near real-time, based on the particle-size distribution predicted by the machine learning model. In other words, a stated benefit of one or more embodiments disclosed herein is that the dosage of the chemicals introduced by a chemical addition unit are continuously adjusted to ensure optimal floc formation and particle aggregation.

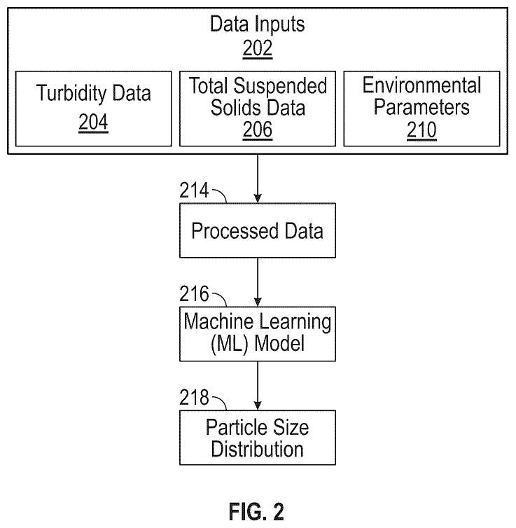

Additionally, the water quality system described herein can generate alerts when the water quality levels reach a lower water quality level than a predefined threshold or standard. Depictions of various configurations of the water quality system and methods of its use are provided in FIGS. 2, 4, 5, 7, and 8, along with accompanying descriptions.

Machine learning, broadly defined, is the extraction of patterns and insights from data. The phrases “artificial intelligence,” “machine learning,” “deep learning,” and “pattern recognition” are often convoluted, interchanged, and used synonymously throughout the literature. This ambiguity arises because the field of “extracting patterns and insights from data” was developed simultaneously and disjointedly among a number of classical arts like mathematics, statistics, and computer science. For consistency, the term machine learning (ML), will be adopted herein, however, one skilled in the art will recognize that the concepts and methods detailed hereafter are not limited by this choice of nomenclature.

Embodiments of the instant disclosure can provide one or more of the following advantages. As will be demonstrated, advantages of the ML-based methods and systems disclosed herein include providing real-time data on particle-size distribution, a significant improvement over traditional methods that rely on periodic manual sampling and laboratory analysis. This allows for immediate detection and response to changes in water quality. Further, the methods and systems disclosed herein offer a higher degree of accuracy and precision in estimating particle sizes compared to conventional turbidity measurements alone. Automating the process of water quality analysis also reduces the need for manual sampling, thereby saving significant labor hours and operational costs. This automation also minimizes human error in data collection and analysis. In addition, with continuous and accurate monitoring, water treatment facilities can optimize their processes more effectively, leading to increased efficiency in water treatment and management. These methods and systems are also designed to integrate with existing turbidity analyzers, thus eliminating the need for significant additional hardware investments. As such, embodiments disclosed herein represent a cost-effective solution for upgrading water quality monitoring systems. Moreover, the ability to closely monitor and control water quality ensures better compliance with environmental regulations and standards, reducing the risk of non-compliance penalties. In addition, the methods and systems disclosed herein are readily scalable to different sizes of water treatment operations and adaptable to various types of water treatment technologies. Likewise, these methods and systems provide detailed data and insights, enabling more informed decision-making in water treatment processes, leading to better resource management and operational strategies. Furthermore, continuous monitoring allows for the collection of long-term data, facilitating trend analysis and predictive maintenance, which can further enhance the efficiency and reliability of water treatment operations. Also, by ensuring efficient and accurate water treatment, these methods and systems contribute to sustainable water management practices, essential in the context of growing environmental concerns and resource conservation. In summary, the ML-based methods and systems disclosed herein offer a comprehensive, efficient, and cost-effective solution for water quality monitoring, addressing many of the limitations of existing methods and significantly enhancing water treatment operations.

FIG. 1 shows a treatment plant in accordance with one or more embodiments. Specifically, FIG. 1 shows a water treatment plant, which is a specialized facility designed to clean and disinfect raw water from natural sources such as rivers, lakes, or groundwater to make it safe for human consumption, industrial use, irrigation, or other purposes. The primary goal of a water treatment plant is to produce clean, potable water that meets specific quality standards and regulatory requirements. It is noted that many types of treatment plants (e.g., water treatment plants) and liquids (e.g., water) exist. Therefore, one with ordinary skill in the art will recognize that any type of treatment plants and liquids may be employed without departing from the scope of this disclosure. Further, it is emphasized that the following discussions of a water treatment plant are basic summaries and should not be considered limiting.

A water treatment plant (100) typically consists of several components designed to purify and process water to make it safe for consumption, or other uses. As shown in FIG. 1, the water treatment plant (100) pumps raw water from a water source (102) (e.g., a lake, a reservoir, etc.). Pumps are used to lift and move water through the treatment process and may be located at various stages of the water treatment plant (100). Typically, the raw water often contains debris such as leaves, sticks, and other large particles. Thus, screens and barriers (not shown) may be used to remove these larger objects to prevent damage to pumps and other downstream equipment. A water sample may be obtained from the water source (102).

The water treatment plant (100) includes a chemical addition unit (104) where chemicals, such as coagulants (e.g., alum) and flocculants (e.g., polymers), are added to the water to facilitate the treatment process and ensure water quality. Mixing and flocculation basins (not shown) may facilitate the mixing of the chemicals with the raw water and promote the formation of flocs, i.e., clusters of suspended particles that can then be easily removed.

In accordance with one or more embodiments, the water treatment plant (100) includes sedimentation tanks (106). These tanks allow suspended solids and flocs to settle to the bottom of the tank, thus producing clearer and cleaner water. Water then passes through a filtration unit (108). The filtration unit (108) includes various layers of filter media (such as sand, gravel, activated carbon, etc.) to remove remaining suspended particles, microorganisms, and other impurities. Further, a disinfection unit (110) uses chemical disinfectants (e.g., chlorine and chloramine) or other treatment methods (e.g., UV disinfection) to kill or deactivate pathogens (e.g., bacteria and viruses) present in the water. Treated water is stored in storage tanks (112) to ensure a continuous and reliable water supply to consumers. A network of pipes, pumps, and valves then distributes the treated water to homes and industries (114). A water treatment plant (100) may also include systems for managing and treating waste generated during the treatment process, such as sludge from sedimentation tanks (106) and spent filter media from the filtration unit (108).

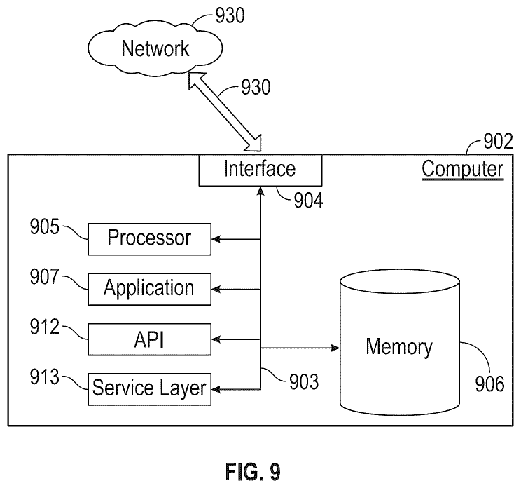

Keeping with FIG. 1, the water treatment plant (100) is monitored by a plurality of sensors (e.g., turbidity meters, particle counters, etc.) and controlled by a control system (“controller”). The controller (105) is communicably connected to the chemical addition unit (104) and controls the dosage of the chemicals introduced by the chemical addition unit (104). These chemicals are designed, among other things, to promote the aggregation of suspended particles into larger flocs, which can then be more easily removed. Aggregation refers to the process by which individual particles come together to form larger clusters (i.e., flocs). As such, the set of dosage parameters of these chemicals affects the kinetics of coagulation and flocculation reactions and thus influences the size of the formed flocs. For example, in some embodiments, higher chemical dosages may lead to larger flocs, resulting in better settling and removal of particles from the water. In other words, the particle-size distribution is controlled, at least in part, by the set of dosage parameters. Therefore, a proper control of the dosage parameters ensures optimal floc formation and settling, and the particle-size distribution may be used, in turn, as a proxy to evaluate the effectiveness of the treatment process. In some embodiments, the controller (105) includes a computer system that controls the chemical addition unit (104), where the computer system is the same as or similar to that of a computer system (902) described below in FIG. 9 and the accompanying description. In one or more embodiments, the controller (105) may be part of the chemical addition unit (104). In other embodiments, the controller (105) may be separate from the chemical addition unit (104).

In some embodiments, the water treatment plant (100) includes the water quality system (116). For example, the water quality system (116) may include hardware and/or software with functionality for determining the particle-size distribution of a water sample and generating alerts indicative of water contamination in the water treatment plant (100). For this purpose, the system may include memory with one or more data structures, such as a buffer, a table, an array, or any other suitable storage medium. In some embodiments, the water quality system (116) may include a computer system similar to the computer system (902) described below with regard to FIG. 9 and the accompanying description. While the water quality system (116) is shown at the water treatment plant (100) in FIG. 1, in some embodiments, the water quality system (116) may be located remotely from the water treatment plant (100).

As previously stated, operation of a water treatment plant (100) can be directed, at least in part, based on a measure of the particle-size distribution of a water sample. As discussed above, traditional methods to determine particle-size involve manual sampling and laboratory analysis. Moreover, manual sampling may be prone to inconsistencies and does not provide real-time data, which is crucial for immediate decision-making in water treatment facilities. As such, a major disadvantage of these methods is that they are often time-consuming, labor-intensive, and do not provide real-time data, which is important for immediate decision-making in water treatment plants. In accordance with one or more embodiments, the particle-size distribution is determined using a ML model, as will be described in greater detail below. Further, and as will be described, the ML is used for the optimization of the water treatment process.

FIG. 2 depicts a flowchart which describes the process of using the ML model to determine the particle-size distribution of a water sample. Initially, data inputs (202) obtained from a plurality of sources are processed to obtain processed data (214). The plurality of sources includes a plurality of sensors (e.g., turbidity meters, particle counters, etc.) appropriately disposed at one or more locations on the water treatment plant (100). Generally, and as will be described later in the instant disclosure, processing comprises, at a minimum, altering the data inputs (202) so that they are suitable for use with ML models. In accordance with one or more embodiments, the data inputs (202) include turbidity data (204), total suspended solids (TSS) data (206), and environmental parameters (210).

Turbidity data (204) refers to measurements or readings that quantify the degree of turbidity. Turbidity refers to the cloudiness or haziness of a fluid caused by large numbers of suspended particles that are generally invisible to the human eye. These particles can include sediment, silt, clay, plankton, microbes, among others. As noted, turbidity data (204) is obtained using turbidity meters, which assess the cloudiness or haziness of the water caused by the suspended particles. These instruments typically measure turbidity by detecting the amount of light scattered by the particles suspended in water. The intensity of the scattered light is directly proportional to the turbidity of the sample. Turbidity is typically measured in Nephelometric Turbidity Units (NTU), Formazin Nephelometric Units (FNU), or Jackson Turbidity Units (JTU) and is an important metric for monitoring water quality, especially in environmental and water treatment contexts. For example, high turbidity levels can affect aquatic life, interfere with disinfection processes in a water treatment plant (100), and reduce the light penetration in aquatic environments. For instance, disinfectants such as chlorine or ozone need sufficient contact time with microorganisms to reach and effectively kill them. High turbidity may reduce the contact time between the disinfectant and the target microorganisms because the suspended particles can adsorb or mitigate the disinfectant, thus reducing its concentration in the water.

TSS data (206) refers to measurements that quantify the amount (i.e., dry-weight) of suspended particles that are not dissolved in water. TSS data (206) is typically collected manually by using water sampling techniques followed by laboratory analysis. Typically, TSS data (206) is obtained by filtering a known volume of water and weighing the suspended solids that remain on the filter. These suspended solids can consist of a variety of materials such as silt, clay, organic matter, inorganic matter, microorganisms, and other particulate substances. TSS data (206) data is often reported in units of milligrams per liter (mg/L) or parts per million (ppm) and indicates the mass of suspended solids present in a given volume of water. High levels of TSS can reduce water clarity and negatively affect aquatic life. Further, in water treatment plants (100), controlling TSS is important for improving the efficiency of filtration and disinfection processes. For instance, reduction of TSS through treatment methods such as sedimentation, filtration, and coagulation may help enhance water clarity, reduce turbidity, and improve the overall quality of the treated water.

In general, turbidity data (204) and TSS data (206) may be related in that they both measure aspects of the particulate content in water. However, they accomplish so in different ways. For example, turbidity quantifies the cloudiness or murkiness in water, influenced by suspended particles that disperse light. On the other hand, TSS quantifies the actual mass of these suspended particles contained in a water sample. In many cases, there is a correlation between turbidity and TSS concentrations. For example, in some embodiments, higher turbidity levels often indicate higher concentrations of TSS in the water, as more particles in the water can scatter more light. However, the relationship is not necessarily direct or linear. In fact, the correlation between turbidity and TSS can be influenced by several factors, including particle-size, particle type, and color. For instance, smaller particles might scatter light more efficiently than larger particles, thus affecting turbidity readings without a proportional increase in the mass of TSS. Further, the composition of the suspended particles (e.g., organic, inorganic) can affect their light scattering properties and how they contribute to turbidity. Similarly, dissolved substances that impart color to the water can also affect turbidity measurements without contributing to TSS. As such, while turbidity is often used as an indicator of water quality and can provide fast insights into changes in suspended solids concentrations, it is not a direct measure of TSS. Therefore, embodiments of the present disclosure include turbidity data (204) and TSS data (206) as independent data inputs (202).

The environmental parameters (210) may include physical properties of the water source (102) such as temperature, pH, dissolved oxygen (DO), conductivity, salinity, nutrient concentrations (e.g., nitrate, phosphate, etc.), among others. Temperature affects water density, viscosity, and the solubility of gases and other substances. In addition, temperature variations can influence the stratification of water bodies and the behavior of chemical reactions. The pH influences the solubility and chemical behavior of pollutants and nutrients in water and can affect the charge and aggregation of particles, which in turn may influence turbidity and TSS. DO levels indicate the presence of organic pollution and also affect oxidation-reduction reactions and the breakdown of substances in the water. Conductivity reflects the water's ability to conduct electricity, which is directly related to the concentration of dissolved ions. As such, conductivity can indicate the overall ionic strength of the water, which may influence particle aggregation and settling. Salinity measures the total concentration of dissolved salts in water (which can affect the density and refractive index of water) and is particularly relevant in coastal and estuarine environments where significant variations are more likely. Nutrients can promote the growth of algae and other microorganisms, thus affecting the composition of suspended particles.

In accordance with one or more embodiments, the turbidity data (204), TSS data (206), and environmental parameters (210) may be obtained in real time or near real time. In some embodiments, the turbidity data (204), TSS data (206), and environmental parameters (210) may be obtained sequentially or immediately after a laboratory analysis is performed. In another embodiments, the turbidity data (204), TSS data (206), and environmental parameters (210) are collected using field devices (i.e., sensors) appropriately disposed at one or more locations on the water treatment plant (100) or obtained from previously collected historical data.

Keeping with FIG. 2, in accordance with one or more embodiments, the data inputs (202) undergo some processing (or pre-processing) in preparation for use with the ML model (216). Processing the data inputs (202) can include data processing procedures such as normalization and imputation, where applicable. In one or more embodiments, processing the data inputs (202) includes organizing and concatenating the data inputs (202), i.e., turbidity data (204), TSS data (206), and environmental parameters (210) such as pH, DO, salinity, etc. in a consistent manner or format. The processed data inputs (202) are referred to as processed data (214). As depicted, the processed data (214) is inputted into the ML model (216) and the ML model (216) outputs the particle-size distribution (218) of the water sample. In some embodiments, no processing (or pre-processing) is applied to the data inputs (202). In such cases, the ML model 216 receives, as an input, the original or raw data inputs (202).

In some embodiments, the ML model (216) determines a particle-size histogram. A particle-size histogram uses intervals or bins to categorize different ranges of particle sizes. These intervals are typically defined by specific size ranges, such as micrometers or nanometers. The height of each bar in the histogram represents the frequency of particles falling within a particular size range. A particle-size histogram provides insights into the characteristics of a sample, including the range of particle sizes present, the presence of any dominant particle-size populations, and the overall distribution pattern (e.g., whether it is normal, skewed, multimodal, etc.). In one or more embodiments, any normality test known in the art may be used to test and/or quantify the normality of the particle-size distribution (218). For instance, the Shapiro-Wilk test, Pearson's chi-squared test, or the Kolmogorov-Smirnov test may be used to test the normality of the particle-size distribution (218) without departing from the scope of this disclosure. In one or more embodiments, the result of the normality test is compared to a user-defined statistical confidence threshold.

In other embodiments, the ML model (216) determines a cumulative distribution function (CDF). The particle-size CDF is defined as the cumulative percentage of particles that are smaller than a given size and ranges from 0% at the smallest particle-size and approaches 100% at the largest particle-size in the water sample. To construct a particle-size CDF, particles are typically sorted into size intervals (i.e., bins), and the cumulative percentage of particles smaller than each size interval is calculated. Specific points on the CDF curve provide information about the percentage of particles that fall below certain critical sizes. For example, the D10, D50 (median particle-size), and D90 values represent the particle sizes at which 10%, 50%, and 90% of particles are smaller, respectively.

In accordance with one or more embodiments, the ML model (216) may determine one or more summary statistical parameters, such as the mean particle-size (indicating the average size of particles in a water sample), median, mode, standard deviation (indicating the spread of particle sizes), kurtosis, or any other suitable summary statistical measures of central tendency and dispersion. For example, in one or more embodiments, the ML model (216) outputs a mean and a variance that parameterize a normal distribution representative of the predicted particle-size distribution. Other distribution assumptions can be used, e.g., a chi-squared distribution or a truncated normal distribution may be deemed more appropriate to avoid the prediction of non-positive particle sizes. As such, in one or more embodiments, the ML model (216) is configured to output the relevant parameters required to define a given distribution (e.g., degrees of freedom for a chi-squared distribution). Predicted distribution parameters can also be used to form or visualize a cumulative distribution function.

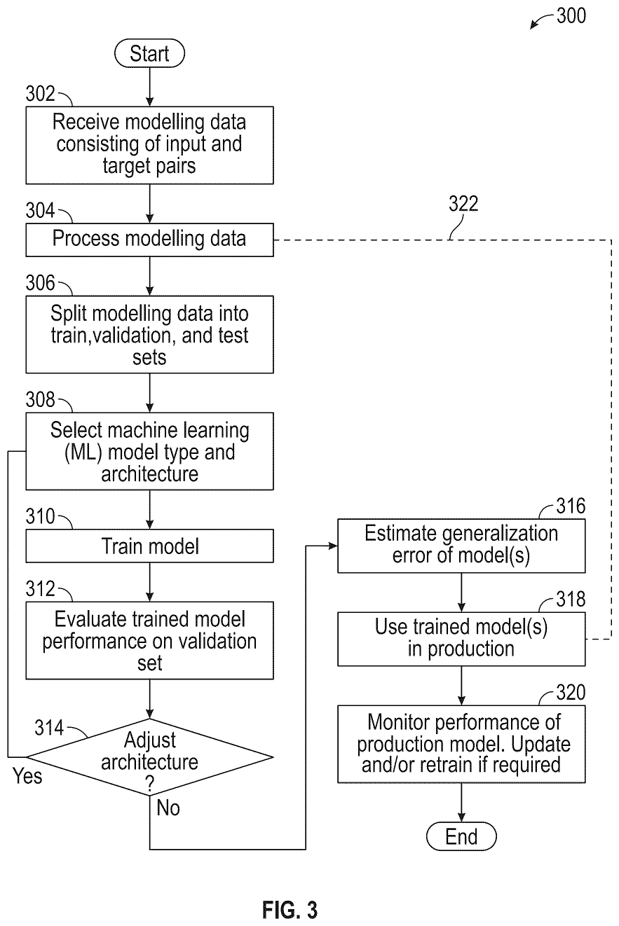

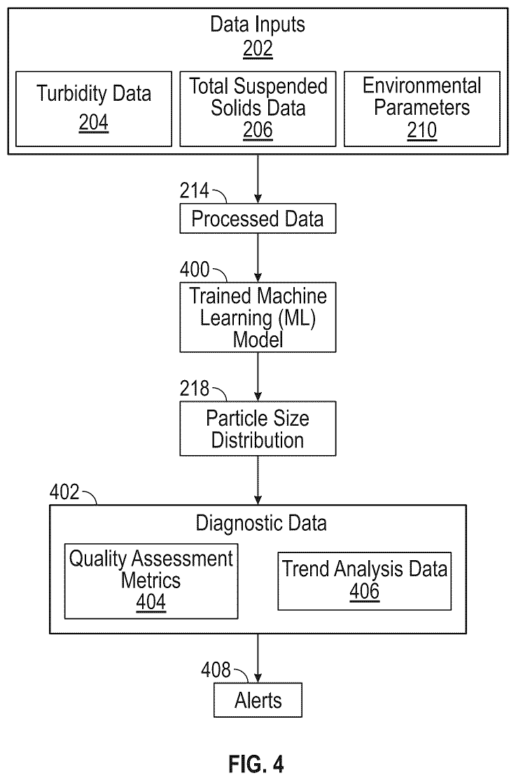

The ML model (216) depicted in FIG. 2 has been trained. FIG. 3 depicts the general process of selecting and training the ML model, in accordance with one or more embodiments. The process shown in FIG. 3 may be applied to obtain the trained ML model discussed with regard to FIGS. 2 and 4 and the accompanying description. To start, as shown in Block 302, modelling data is received. The modelling data consists of input and target pairs. For example, to train the ML model, an input and target pair may consist of data inputs (202) and a known associated particle-size distribution (218) or summary statistic. That is, a training pair included in the modelling data consists of an input (e.g., data inputs (202)) and a known target (e.g., particle-size distribution, summary statistic). As will be described below, during training the known target is compared to a predicted output of the ML model processing the associated input. The comparison guides the training process. In accordance with one or more embodiments, data inputs (202) include turbidity data (204), TSS data (206), and environmental parameters (210). In one or more embodiments, the turbidity data (204), TSS data (206), environmental parameters (210), and associated targets are obtained from previously collected historical data.

Keeping with FIG. 3, in one or more embodiments, the modelling data is processed as depicted by Block 304. Processing, at a minimum, includes altering the modelling data so that it is suitable for use with ML models. For example, numericalizing categorical data or removing data entries with missing values. Other typical processing methods are normalization and imputation. Information surrounding the processing steps is saved for potential later use. For example, if normalization is performed then a computed mean vector and variance vector are retained. This allows future modelling data to be processed identically. For example, the processed data (214) as previously depicted in FIG. 2, will have undergone the same processing steps developed with respect to Block 304. Values computed and retained during processing are referred to herein as processing parameters. One with ordinary skill in the art will recognize that a myriad of processing methods beyond numericalization, removal of modelling data entries with missing values, normalization, and imputation exist. Descriptions of a select few processing methods herein do not impose a limitation on the processing steps encompassed by this disclosure.

As shown in Block 306, the modelling data is split into training, validation, and test sets. In some embodiments, the validation and test set may be the same such that the data is effectively only split into two distinct sets. In some instances, Block 306 may be performed before Block 304. In this case, it is common to determine the processing parameters, if any, using the training set and then to apply these parameters to the validation and test sets.

In Block 308, the ML model type and associated architecture is selected. Once selected, the ML model is trained using the training set of the modelling data according to Block 310. Common training techniques, such as early stopping, adaptive or scheduled learning rates, and cross-validation may be used during training without departing from the scope of this disclosure.

ML model types may include, but are not limited to, support vector machines, K-means clustering, K-nearest neighbors, neural networks, logistic regression, random forests, generalized linear models, and Bayesian regression. ML models may make use of fuzzy logic or otherwise process values and produce results that are non-binary. For example, in the present context, the ML model may make use of or produce a representation indicative of a degree of cloudiness of a water sample as opposed to an indication that the water sample is clear or cloudy. Also, ML encompasses model types that may further be categorized as “supervised,” “unsupervised,” “semi-supervised,” or “reinforcement” models. One with ordinary skill in the art will appreciate that additional or alternate ML model categorizations may be defined without departing from the scope of this disclosure. Constraining a model to make it simpler and reduce the risk of overfitting is called regularization. The amount of regularization to be applied during learning may be controlled by “hyperparameters” which further describe the ML model. For example, hyperparameters providing further detail about a neural network may include, but are not limited to, the number of layers in the neural network, choice of activation functions, inclusion of batch normalization layers, and regularization strength. Commonly, in the literature, the selection of hyperparameters surrounding a model is referred to as selecting the model “architecture.” Generally, multiple model types and associated hyperparameters are tested and the model type and hyperparameters that yield the greatest predictive performance on a hold-out set of data is selected.

During training, or once trained, the performance of the trained ML model is evaluated using the validation set as depicted in Block 312. Recall that, in some instances, the validation and test sets are the same. Generally, performance is measured using a function which compares the predictions of the trained ML model to the given targets. A commonly used comparison function is the mean-squared-error function, which quantifies the difference between the predicted value and the actual value when the predicted value is continuous. However, one with ordinary skill in the art will appreciate that many more comparison functions exist and may be used without limiting the scope of the present disclosure. For example, a comparison of a predicted particle-size distribution and a known or target particle-size distribution can be performed using the cross-entropy function.

At Block 314, a determination is made as to whether the ML model architecture needs to be altered. If the trained ML model performance, as measured by a comparison function on the validation set (Block 312), is suitable, then the trained ML model is accepted for use in a production setting. As such, in Block 318, the trained ML model is used in production. However, before the ML model is used in production, a final indication of its performance can be acquired by estimating the generalization error of the trained ML model, as shown in Block 316. Generalization error is an indication of the trained ML model's performance on new, or un-seen data. Typically, the generalization error is estimated using the comparison function, as previously described, using the modelling data that was partitioned into the test set.

At Block 314, if the trained ML model performance is not suitable, the ML model architecture may be altered (i.e., return to Block 308) and the training process is repeated. There are many ways to alter the ML model architecture in search of suitable trained ML model performance. These include, but are not limited to, selecting a new architecture from a previously defined set; randomly perturbing or randomly selecting new hyperparameters; using a grid search over the available hyperparameters; and intelligently altering hyperparameters based on the observed performance of previous models (e.g., a Bayesian hyperparameter search). Once suitable performance is achieved, the training procedure is complete, and the generalization error of the trained ML model is estimated according to Block 316.

As depicted in Block 318, the trained ML model is used “in production,” which means that the trained ML model is used to process a received input without having a paired target for comparison. It is emphasized that the inputs received in the production setting, as well as for the validation and test sets, are processed identically to the manner defined in Block 304 as denoted by the connection (322), represented as a dashed line in FIG. 3, between Blocks 318 and 304.

In accordance with one or more embodiments, the performance of the trained ML model is continuously monitored in the production setting (320). If model performance is suspected to be degrading, as observed through in-production performance metrics, the model may be updated. An update may include retraining the model, by reverting to Block 308, with the newly acquired modelling data from the in-production recorded values appended to the training data. An update may also include recalculating any processing parameters, again, after appending the newly acquired modelling data to the existing modelling data.

While the various blocks in FIG. 3 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

The process of using the trained ML model (400) “in production” is shown in the flowchart of FIG. 4. FIG. 4 differs from FIG. 2 in that it additionally demonstrates the use of the predicted particle size distribution (i.e., determined using the ML model) in a more encompassing system, namely, a water quality system in accordance with one or more embodiments. Further, to emphasize that the flowchart of FIG. 4 uses the ML model after training, it is referred to as the trained ML model (400). As discussed, the trained ML model (400) is used to process a received input without having a paired target for comparison. Turning to FIG. 4, data inputs (202) are received as inputs by the trained ML model (400). The ML model inputs (202) will be of the same form as the inputs used during training and thus may include turbidity data (204), TSS data (206), and environmental parameters (210). In accordance with one or more embodiments, the trained ML model (400) outputs an ML predicted particle-size distribution (218). Descriptions of the data inputs (202) and various formats for the output particle-size distribution (218) were previously discussed with respect to FIG. 2 and are not repeated here for concision.

In accordance with one or more embodiments, the trained ML model (400) processes data inputs (202) acquired using field devices (e.g., sensors) appropriately disposed at one or more locations on the water treatment plant (100). For example, the turbidity data (204) may represent actual measurements of turbidity as determined, for example, by a turbidity meter. In such an embodiment, the trained ML model (400) may process its input in real time, or near real time, such that the particle-size distribution (218) may be determined using only turbidity data (204).

In accordance with one or more embodiments, the particle-size distribution (218) determined by the trained ML model (400) is used to provide diagnostic data (402). Examples of diagnostic data (402) are shown in FIG. 4. For example, diagnostic data (402) includes quality assessment metrics (404) and trend analysis data (406). Quality assessment metrics (404) encompass a range of physical, chemical, and biological parameters that collectively indicate the water's quality and suitability for various uses, such as drinking, industrial processes, irrigation, and recreational activities. For example, in some embodiments, the quality assessment metrics (404) includes a water quality level, which indicates if the presence of bacteria, microorganisms, and algae is detected in a water sample. Quality assessment metrics (404) are essential for monitoring water quality and ensuring it meets the standards required for its intended use. Regulatory bodies often set limits on these parameters to protect human health and the environment. Further, trend analysis data (406) may include water quality reports that summarize water quality trends, anomalies, and overall system performance. For instance, these water quality reports may be generated over time based on previously recorded and analyzed events to support an effective decision making process. Accumulated data over time can also reveal trends in water quality changes, thus assisting in long-term planning, infrastructure development, and investment decisions to address future water treatment needs. For conciseness, not all diagnostic data (402) are enumerated in FIG. 4. However, one with ordinary skill in the art will recognize that many alterations to the diagnostic data (402) of FIG. 4 may be made without departing from the scope of this disclosure.

Keeping with FIG. 4, in one or more embodiments, once the diagnostic data (402) has been determined, one or more alerts indicative of water contamination are generated. In such an embodiment, operators (e.g., water treatment plant operators) receive alerts if significant changes in water quality are detected, and can have access detailed reports (e.g., water quality reports) that include particle-size distribution and other relevant metrics to take appropriate action. In some embodiments, one or more alerts (e.g., visual warnings) are presented to operators in real-time using an output device (e.g., a dashboard). The operators may then be able to act based on the information presented on the output device. For example, in one or more embodiments, an operator may determine a threshold and the quality assessment metrics (404) (e.g., water quality levels) may be continuously compared to the threshold. For instance, the water quality levels that are equal to or higher than the threshold may raise a flag and alert an operator that the water may be contaminated. Alternatively, the water quality levels that are lower than the threshold may not raise a flag, and the treatment process continues to operate without interruptions.

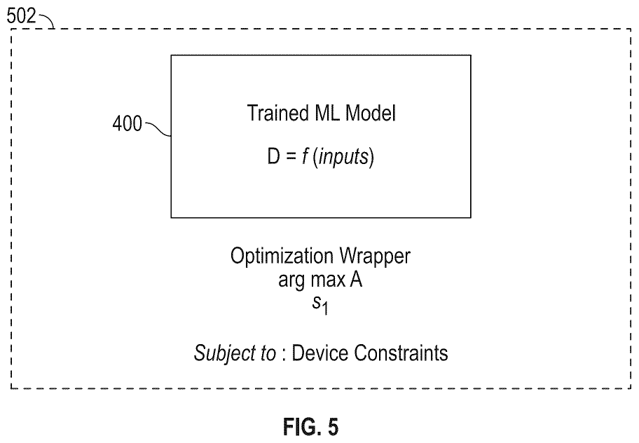

The process of using the trained ML model (400) to determine the optimal set of dosage parameters that optimize particle aggregation is summarized in FIG. 5. In one or more embodiments, the ML model is trained using previously acquired modelling date, e.g., historical particle-size distribution data. As previously described, the modelling data is partitioned into “inputs” and “targets.” The targets include particle-size distribution (218) or a suitable representation (e.g., relevant distribution parameter(s)). The inputs include turbidity data (204), TSS data (206), and environmental parameters (210). The modelling data, partitioned into inputs and targets, are used to train the ML model (216). The trained ML model (400) may be of any type known in the art. Generally, the ML model type and architecture with the greatest performance on a set of hold-out data is selected. Greater detail surrounding the training procedure for the ML model will be provided below in the context of a neural network. However, generally, training the ML model involves processing data to develop a functional relationship between elements of the data. The result of the training procedure is a trained ML model (400). The trained ML model may be described as a function relating the inputs and the outputs. That is, the ML model may be mathematically represented as outputs=ƒ(inputs), such that, given an input, the trained ML model (400) may produce an output. In one or more embodiments, the trained ML model upon processing an input produces an output, namely, a particle-size distribution (D). With a trained ML model, an optimization wrapper (depicted as Block 502) is used to invert the model to determine the optimal set of dosage parameters that optimize particle aggregation. Mathematically, the optimization takes the form:

$${arg max}_{{S}_{1}} A \qquad Equation ( 1 )$$

$$subject to : device constraints ,$$

where the quantity A represents the particle aggregation. Further, in EQ. 1, the set of dosage parameters is denoted as S₁.

As previously stated, the particle-size distribution (218) as determined using the trained ML model (400) is controlled, at least in part, by the set of dosage parameters. In addition, the set of dosage parameters determine the dosage of the chemicals introduced by the chemical addition unit (104) which, in turn, controls particle aggregation. Therefore, the particle-size distribution (218) is affected by changes in particle aggregation. For example, in some embodiments, the particle-size distribution (218) may shift towards larger sizes as larger particles form. In other embodiments, aggregation may alter the shape of the particle-size distribution (218) and lead to a broader distribution with a higher proportion of larger particles. In another embodiment, aggregates or agglomerates formed due to particle aggregation contribute differently to the particle-size distribution (218) compared to individual primary particles and can thus lead to a multimodal distribution, especially if there are individual particles and aggregates simultaneously present. Accordingly, the dosage of the chemicals introduced by the chemical addition unit (104) may be continuously adjusted to maximize particle aggregation over the set of dosage parameters in accordance with one or more embodiments of the present disclosure.

The optimization wrapper (502), when applied to a trained ML model parameterized by the set of dosage parameters, returns a single and optimal set of dosage parameters. Optimization algorithms may include, but are not limited to, genetic, Newton conjugate gradient (Newton-CG), Broyden-Fletcher-Goldfarb-Shanno (BFGS), and limited-memory BFGS (L-BFGS) algorithms.

One with ordinary skill in the art will appreciate that maximization and minimization may be made equivalent through simple techniques such as negation. As such, the choice to represent the optimization as a maximization as shown in EQ. 1 does not limit the scope of the present disclosure. Whether done through minimization or maximization, the optimization wrapper (502) identifies the set (or sets) of dosage parameters that optimize particle aggregation according to the trained ML model (400).

A water treatment plant (100) may be subject to constraints, such as treatment process limitations. For example, each treatment process (e.g., coagulation, sedimentation, filtration, etc.) has specific capabilities and limitations in terms of particle removal efficiency and size range. As such, constraints may arise from the practical limitations of existing technologies and infrastructure. For instance, in some embodiments, coagulation and flocculation processes used to aggregate suspended particles into larger flocs may be limited if the coagulant dosage is insufficient to achieve effective particle destabilization and floc formation, particularly for certain types of organic or low-density particles. In other embodiments, certain types of filters may struggle to adequately remove very fine particles, thus leading to a reduced treatment efficiency. For example, filtration processes such as rapid sand filtration or membrane filtration have limitations on the particle-size range that they can effectively remove. In FIG. 5, these constraints are referenced to as device constraints. Accordingly, the optimization wrapper (502) cannot elect any set of dosage parameters that cause any treatment process in the water treatment plant (100) to exceed predefined constraints. Additional examples of constraints applied to the optimization may include predefined energy consumption and overdosing limitations, which can result in undesirable chemical residuals and increased operational costs.

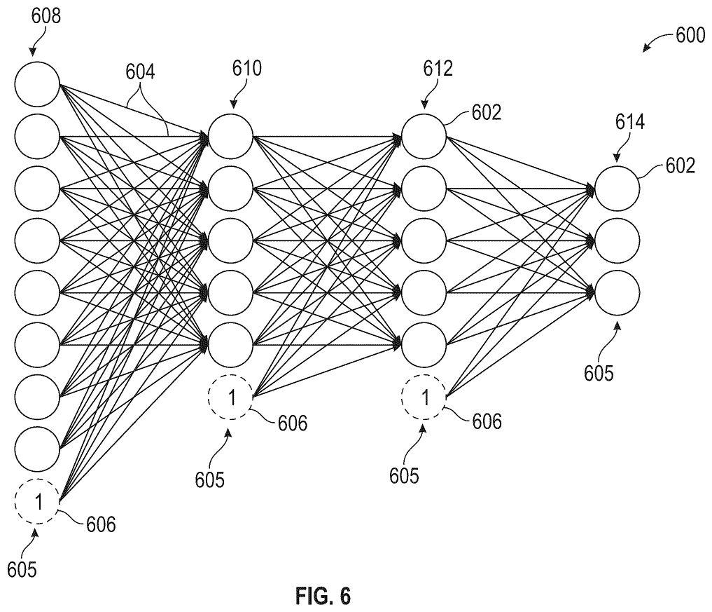

In accordance with one or more embodiments, the ML model discussed herein is a neural network. A diagram of a neural network is shown in FIG. 6. At a high level, a neural network (600) may be graphically depicted as being composed of nodes (602), where here any circle represents a node, and edges (604), shown here as directed lines. The nodes (602) may be grouped to form layers (605). FIG. 6 displays four layers (608, 610, 612, 614) of nodes (602) where the nodes (602) are grouped into columns, however, the grouping need not be as shown in FIG. 6. The edges (604) connect the nodes (602). Edges (604) may connect, or not connect, to any node(s) (602) regardless of which layer (605) the node(s) (602) is in. That is, the nodes (602) may be sparsely and residually connected. A neural network (600) will have at least two layers (605), where the first layer (608) is considered the “input layer” and the last layer (614) is the “output layer.” Any intermediate layer (610, 612) is usually described as a “hidden layer”. A neural network (600) may have zero or more hidden layers (610, 612) and a neural network (600) with at least one hidden layer (610, 612) may be described as a “deep” neural network or as a “deep learning method.” In general, a neural network (600) may have more than one node (602) in the output layer (614). In this case the neural network (600) may be referred to as a “multi-target” or “multi-output” network.

Nodes (602) and edges (604) carry additional associations. Namely, every edge is associated with a numerical value. The edge numerical values, or even the edges (604) themselves, are often referred to as “weights” or “parameters.” While training a neural network (600), numerical values are assigned to each edge (604). Additionally, every node (602) is associated with a numerical variable and an activation function. Activation functions are not limited to any functional class, but traditionally follow the form

$$A = f ( {Σ}_{i ∈ ( incoming )} [ {( node value )}_{i} {( edge value )}_{i} ] ) \qquad Equation ( 2 )$$

where i is an index that spans the set of “incoming” nodes (602) and edges (604) and ƒ is a user-defined function. Incoming nodes (602) are those that, when viewed as a graph (as in FIG. 6), have directed arrows that point to the node (602) where the numerical value is being computed. Some functions for ƒ may include the linear function ƒ(x)=x, sigmoid function

$$f ( x ) = \frac{1}{1 + {e}^{- x}} ,$$

and rectified linear unit function ƒ(x)=max(0, x), however, many additional functions are commonly employed. Every node (602) in a neural network (600) may have a different associated activation function. Often, as a shorthand, activation functions are described by the function ƒ by which it is composed. That is, an activation function composed of a linear function ƒ may simply be referred to as a linear activation function without undue ambiguity.

When the neural network (600) receives an input, the input is propagated through the network according to the activation functions and incoming node (602) values and edge (604) values to compute a value for each node (602). That is, the numerical value for each node (602) may change for each received input. Occasionally, nodes (602) are assigned fixed numerical values, such as the value of 1, that are not affected by the input or altered according to edge (604) values and activation functions. Fixed nodes (602) are often referred to as “biases” or “bias nodes” (606), displayed in FIG. 6 with a dashed circle.

In some implementations, the neural network (600) may contain specialized layers (605), such as a normalization layer, or additional connection procedures, like concatenation. One skilled in the art will appreciate that these alterations do not exceed the scope of this disclosure.

As noted, the training procedure for the neural network (600) comprises assigning values to the edges (604). To begin training the edges (604) are assigned initial values. These values may be assigned randomly, assigned according to a prescribed distribution, assigned manually, or by some other assignment mechanism. Once edge (604) values have been initialized, the neural network (600) may act as a function, such that it may receive inputs and produce an output. As such, at least one input is propagated through the neural network (600) to produce an output. Recall, that a given data set will be composed of inputs and associated target(s), where the target(s) represent the “ground truth,” or the otherwise desired output.

The neural network (600) output is compared to the associated input data target(s). The comparison of the neural network (600) output to the target(s) is typically performed by a so-called “loss function;” although other names for this comparison function such as “error function,” “misfit function,” and “cost function” are commonly employed. Many types of loss functions are available, such as the mean-squared-error function, however, the general characteristic of a loss function is that the loss function provides a numerical evaluation of the similarity between the neural network (600) output and the associated target(s). The loss function may also be constructed to impose additional constraints on the values assumed by the edges (604), for example, by adding a penalty term, which may be physics-based, or a regularization term. Generally, the goal of a training procedure is to alter the edge (604) values to promote similarity between the neural network (600) output and associated target(s) over the data set. Thus, the loss function is used to guide changes made to the edge (604) values, typically through a process called “backpropagation.”

While a full review of the backpropagation process exceeds the scope of this disclosure, a brief summary is provided. Backpropagation consists of computing the gradient of the loss function over the edge (604) values. The gradient indicates the direction of change in the edge (604) values that results in the greatest change to the loss function. Because the gradient is local to the current edge (604) values, the edge (604) values are typically updated by a “step” in the direction indicated by the gradient. The step size is often referred to as the “learning rate” and need not remain fixed during the training process. Additionally, the step size and direction may be informed by previously seen edge (604) values or previously computed gradients. Such methods for determining the step direction are usually referred to as “momentum” based methods.

Once the edge (604) values have been updated, or altered from their initial values, through a backpropagation step, the neural network (600) will likely produce different outputs. Thus, the procedure of propagating at least one input through the neural network (600), comparing the neural network (600) output with the associated target(s) with a loss function, computing the gradient of the loss function with respect to the edge (604) values, and updating the edge (604) values with a step guided by the gradient, is repeated until a termination criterion is reached. Common termination criteria are: reaching a fixed number of edge (604) updates, otherwise known as an iteration counter; a diminishing learning rate; noting no appreciable change in the loss function between iterations; reaching a specified performance metric as evaluated on the data or a separate hold-out data set. Once the termination criterion is satisfied, and the edge (604) values are no longer intended to be altered, the neural network (600) is said to be “trained.”

In one or more embodiments, the ML model discussed herein is a support vector machine. In general, a support vector machine regressor may be decomposed into two parts. First, a support vector machine regressor transforms the input data to a feature space. The feature space is usually a higher dimensional space than the space of the original input data. The transformation is performed using a function from a family of functions often referred to in the literature as “kernel” functions. Many kernel functions exist and kernel functions may be created, usually through a combination of other kernel functions, according to a specific use-case. The choice of kernel function for a support vector machine regressor is a hyperparameter of the support vector machine model. Kernel functions possess certain mathematical properties. While a complete description of kernel functions and their associated properties exceeds the scope of this disclosure, it is stated that an important property of kernel functions is that they are amenable to the so-called “kernel trick.” The kernel trick allows for distances to be computed between pairs of data points in the feature space without actually transforming the data points from the original input space to the feature space. The second part of a support vector machine consists of parameterizing a hyperplane in the feature space. The hyperplane is described by a set of weights, {w₀, w₁, . . . , wₙ}. The hyperplane represents the predicted value of the support vector machine regressor given an input and can be written as

$$y = {w}_{0} + {\sum }_{i = 1}^{n} {w}_{i} {x}_{i} , \qquad Equation ( 3 )$$

where y is the value of the hyperplane and xᵢ is a value on an axis i of the feature space where the feature space has n dimensions. Note that in some implementations a support vector machine regressor and associated kernel, the weight w₀ may be included in the summation. The set of weights may be described using a vector w. Likewise, a data point in the feature space may be described as a vector x. Incorporating w₀ into the weight vector and using vector notation, the prediction for a data point indexed by j may be written as

$${y}_{j} = {w}^{T} {x}_{j} . \qquad Equation ( 4 )$$

To determine the values of the weights for a support vector machine regressor, also known as training the support vector machine model, the following optimization problem is solved:

$$min \frac{1}{2} {‖ w ‖}^{2} \qquad Equation ( 5 )$$

$$subject to : \vert {y}_{j} - {w}^{T} {x}_{j} \vert \leq ϵ , ∀ j in training data ,$$

where ϵ is an error term, set by the user and may be considered another hyperparameter of the support vector machine model. From EQ. 4, it is seen that w^{T}xⱼ represents the predicted value, or in the context of the present disclosure, the predicted gas flow rate, for a training data point xⱼ. As such, the constraint |yⱼ−w^{T}xⱼ|≤ϵ in EQ. 5 indicates that the difference between the actual value yⱼ and the predicted value w^{T}xⱼ must be smaller than some pre-defined error ϵ. While this is an acceptable practice, it is noted that the hyperplane determined by EQ. 5 is quite sensitive to outlier data values. This is because the entirety of the hyperplane may need to be altered, often adversely, in order to accommodate the constraint of EQ. 5 for an outlier data point, or the value of ϵ may have to be increased. To mitigate the negative effects of outliers in the data, and more generally to produce a support vector machine regressor with greater predictive power, EQ. 5 is altered to included slack terms ξⱼ and a regularization term λ as follows:

$$min ( \frac{1}{2} {‖ w ‖}^{2} + λ {\sum }_{j = 1}^{m} \vert {ξ}_{j} \vert ) \qquad Equation ( 6 )$$

$$subject to : \vert {y}_{j} - {w}^{T} {x}_{j} \vert \leq ϵ + \vert {ξ}_{j} \vert , ∀ j .$$

In EQ. 6, there are m data points in the training set and the data points are indexed by j. For each training data point there is a slack term ξⱼ which can alleviate the constraint. As such, the constraint may be satisfied, for example, for outlier data points, without altering the hyperplane. If the slack terms were allowed to grow without limitation, the slack terms would obviate the constraint. To counter this, the slack terms are preferred to be kept at minimal values as demonstrated by the second term to be minimized,

$${Σ}_{j = 1}^{m} \vert {ξ}_{j} \vert .$$

The inclusion of the second term in the minimization operator introduces a tradeoff between adjusting the hyperplane and limiting the slack terms. This tradeoff is controlled by the regularization term λ, which may be considered a hyperparameter of the support vector machine model.

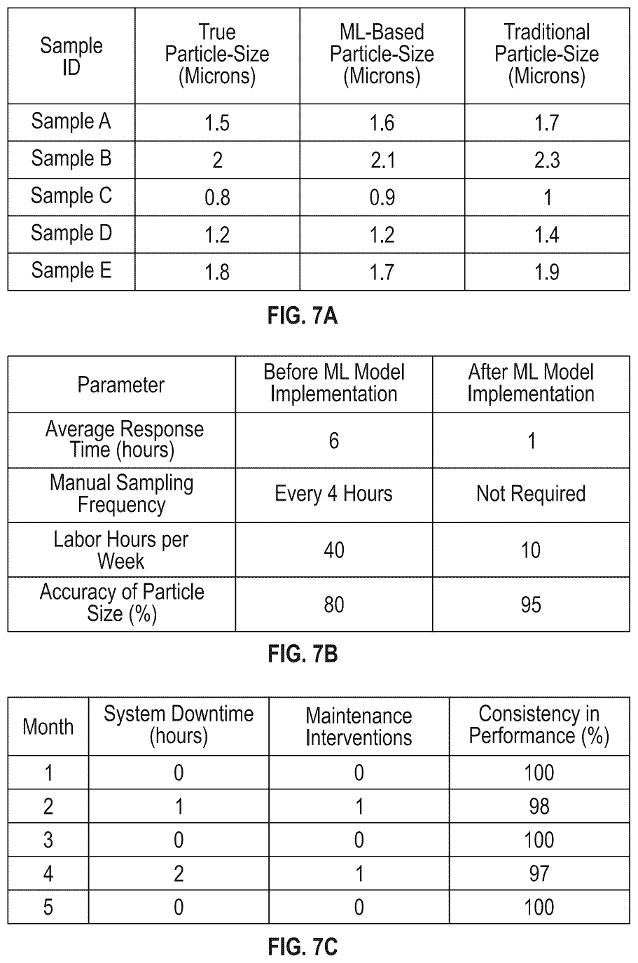

As a concrete example, FIGS. 7A-7C show the results of a pilot implementation of the workflow of FIG. 4 in a water treatment plant (100). Specifically, FIGS. 7A-7C focus on parameters such as accuracy, efficiency, and operational impact, respectively. FIG. 7A shows a comparison between mean values of the true particle-size, the ML-based estimation of the particle-size (“ML-based particle-size”) and the particle-size obtained using traditional methods (“traditional particle-size”) for five different water samples, labeled Sample A through E, in units of micrometers (i.e., microns). The true particle-size represents the particle size as determined through precise laboratory measurements. This value serves as a benchmark for assessing the accuracy of the other two particle-size estimation methods. The ML-based estimation of the particle-size is obtained using the trained ML model (400). The traditional particle-size is estimated using traditional methods and involves manual sampling and subsequent laboratory analysis. In all five water samples shown in FIG. 7A, the ML model shows closer estimations to the true particle size as compared to traditional methods, thus indicating a higher prediction accuracy. FIG. 7B shows an operational efficiency analysis before and after the use of the ML model. As seen in FIG. 7B, the implementation of the ML model leads to an improvement in efficiency, such as reduced response times, reduced labor hours, and increased accuracy in the particle-size detection. FIG. 7C depicts month-by-month comparisons of key performance indicators, including system downtime, maintenance interventions, and performance consistency. As shown, the pilot implementation of the ML model demonstrates high reliability with minimal downtime and consistent performance over a 5-month period.

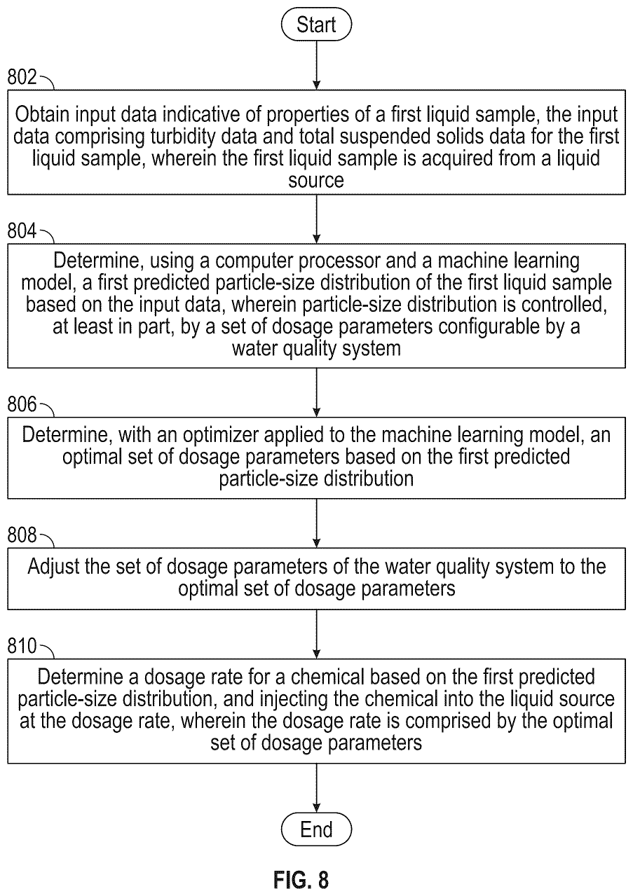

FIG. 8 depicts a method for water quality assessment, in accordance with one or more embodiments. In Block 802, property data inputs (202) (“input data”) from one or more sources are obtained. The property data inputs (202) are indicative of properties of a liquid (e.g., water) sample acquired from a liquid source (e.g., a water source (102) such as a lake, a reservoir, etc.). The property data inputs (202) include turbidity data (204) and TSS data (206). The one or more sources include a plurality of sensors (e.g., turbidity meters, particle counters, etc.) appropriately disposed at one or more locations on a water treatment plant (100). Turbidity refers to the cloudiness or haziness of a fluid caused by large numbers of suspended particles that are generally invisible to the human eye. These particles can include, for example, sediment, silt, clay, plankton, microbes, among others. As noted, turbidity data (204) refers to measurements or readings that quantify the degree of turbidity. Turbidity data (204) is obtained using, for example, turbidity meters, which assess the cloudiness or haziness of the water caused by the suspended particles. High turbidity levels can affect aquatic life, interfere with disinfection processes in a water treatment plant (100), and reduce the light penetration in aquatic environments. On the other hand, TSS data (206) refers to measurements that quantify the amount (i.e., dry-weight) of suspended particles that are not dissolved in water. TSS data is typically collected manually by using water sampling techniques followed by laboratory analysis. Specifically, TSS is obtained by filtering a known volume of water and weighing the suspended solids that remain on the filter. These suspended solids can consist of a variety of materials such as, for example, silt, clay, organic matter, inorganic matter, microorganisms, and other particulate substances. High levels of TSS can reduce water clarity and negatively affect aquatic life. Further, in water treatment plants (100), controlling TSS is important for improving the efficiency of filtration and disinfection processes. For instance, reduction of TSS through treatment methods such as sedimentation, filtration, and coagulation may help enhance water clarity, reduce turbidity, and improve the overall quality of the treated water.

In Block 804, the particle-size distribution (218) of the liquid (e.g., water) sample is predicted using a trained ML model (400) based on the property data inputs (202). In some embodiments, the trained ML model (400) determines a particle-size histogram. A particle-size histogram provides insights into the characteristics of a sample, including the range of particle sizes present, the presence of any dominant particle-size populations, and the overall distribution pattern (e.g., whether it is normal, skewed, multimodal, etc.). In one or more embodiments, any normality test known in the art may be used to test and/or quantify the normality of the particle-size distribution (218). For instance, the Shapiro-Wilk test, Pearson's chi-squared test, or the Kolmogorov-Smirnov test may be used to test the normality of the particle-size distribution (218) without departing from the scope of this disclosure. In one or more embodiments, the result of the normality test is compared to a user-defined statistical confidence threshold. In other embodiments, the trained ML model (400) determines a cumulative distribution function (CDF). The particle-size CDF is defined as the cumulative percentage of particles that are smaller than a given size. Specific points on the CDF curve provide information about the percentage of particles that fall below certain critical sizes. For example, the D10, D50 (median particle-size), and D90 values represent the particle sizes at which 10%, 50%, and 90% of particles are smaller, respectively. In accordance with one or more embodiments, the trained ML model (400) may determine one or more summary statistical parameters, such as the mean particle-size (indicating the average size of particles in a water sample), median, mode, standard deviation (indicating the spread of particle sizes), kurtosis, or any other suitable summary statistical measures of central tendency and dispersion.

Keeping with Block 804, the water treatment plant (100) is controlled by a control system (“controller”) and/or water quality system. The controller (105) is communicably connected to the chemical addition unit (104) and controls the chemicals introduced by the chemical addition unit (104). In one or more embodiments, the controller (105) may be part of the chemical addition unit (104). In other embodiments, the controller (105) may be separate from the chemical addition unit (104). The chemicals introduced by the chemical addition unit (104) are designed to promote the aggregation of suspended particles into larger flocs, which can then be more easily removed. Aggregation refers to the process by which individual particles come together to form larger clusters (i.e., flocs). As such, the particle-size distribution is controlled, at least in part, by the set of dosage parameters. Therefore, a proper control of the dosage parameters ensures optimal floc formation and settling, and the particle-size distribution (218) may be used, in turn, as a proxy to evaluate the effectiveness of the treatment process.

In Block 806, an optimal set of dosage parameters is determined with an optimizer applied to the ML model. In one or more embodiments, the ML model is trained using previously acquired modelling date, e.g., historical particle-size distribution data. In one or more embodiments, the trained ML model, upon processing an input (e.g., data inputs (202)) produces an output, namely, a particle-size distribution (D). With a trained ML model, an optimization wrapper is used to invert the model to determine the optimal set of dosage parameters that optimize particle aggregation. As previously stated, the particle-size distribution (218) is controlled, at least in part, by the set of dosage parameters. In addition, the set of dosage parameters determine the dosage of the chemicals introduced by the chemical addition unit (104) which, in turn, controls particle aggregation. Therefore, the particle-size distribution (218) is affected by changes in particle aggregation. For example, in some embodiments, the particle-size distribution (218) may shift towards larger sizes as larger particles form. In other embodiments, aggregation may alter the shape of the particle-size distribution (218) and lead to a broader distribution with a higher proportion of larger particles. In another embodiment, aggregates or agglomerates formed due to particle aggregation contribute differently to the particle-size distribution (218) compared to individual primary particles and can thus lead to a multimodal distribution, especially if there are individual particles and aggregates simultaneously present. Accordingly, the dosage of the chemicals introduced by the chemical addition unit (104) may be continuously adjusted to maximize particle aggregation over the set of dosage parameters in accordance with one or more embodiments of the present disclosure.

In Block 808, the optimization wrapper (502), when applied to a trained ML model parameterized by the set of dosage parameters, returns a single and optimal set of dosage parameters. Accordingly, the controller (105) adjusts the set of dosage parameters to the optimal set of dosage parameters. Optimization algorithms may include, but are not limited to, genetic, Newton conjugate gradient (Newton-CG), Broyden-Fletcher-Goldfarb-Shanno (BFGS), and limited-memory BFGS (L-BFGS) algorithms. A water treatment plant (100) may be subject to constraints, such as treatment process limitations. For example, each treatment process (e.g., coagulation, sedimentation, filtration, etc.) has specific capabilities and limitations in terms of particle removal efficiency and size range. As such, constraints may arise from the practical limitations of existing technologies and infrastructure. Therefore, the optimization wrapper (502) cannot elect any set of dosage parameters that cause any treatment process in the water treatment plant (100) to exceed predefined constraints.

In Block 810, a chemical dosage rate is determined based on the particle-size distribution (218) of the liquid sample. As noted, the water treatment plant (100) is controlled by a control system (“controller”). The controller (105) is communicably connected to the chemical addition unit (104) and controls the chemicals introduced by the chemical addition unit (104). As such, the controller (105) is configured to adjust a set of dosage parameters of one or more chemicals used by the water quality system (116). Thus, in Block 810, the controller (105) injects a chemical (e.g., a coagulant, a flocculant, etc.) into the liquid source (e.g., a water source (102) such as a lake, a reservoir, etc.) at the dosage rate. The optimal set of dosage parameters include the dosage rate. In some embodiments, the controller (105) includes a computer system that controls the chemical addition unit (104), where the computer system is the same as or similar to that of a computer system (902) described below in FIG. 9 and the accompanying description.

Embodiments disclosed herein may be implemented on a computer system. FIG. 9 is a block diagram of a computer system (902) used to provide computational functionalities associated with described algorithms, methods, functions, processes, flows, and procedures as described in the instant disclosure, according to one or more embodiments. For example, the controller (105) of the water quality system (116) can include, or can be, a computer system (902) such as that depicted in FIG. 9. The illustrated computer (902) is intended to encompass any computing device such as a server, desktop computer, laptop/notebook computer, wireless data port, smart phone, personal data assistant (PDA), tablet computing device, one or more processors within these devices, or any other suitable processing device such as an edge computing device, including both physical or virtual instances (or both) of the computing device. An edge computing device is a dedicated computing device that is, typically, physically adjacent to the process or control with which it interacts.

Additionally, the computer (902) may include a computer that includes an input device, such as a keypad, keyboard, touch screen, or other device that may accept user information, and an output device that conveys information associated with the operation of the computer (902), including digital data, visual, or audio information (or a combination of information), or a GUI.

The computer (902) may serve in a role as a client, network component, a server, a database or other persistency, or any other component (or a combination of roles) of a computer system for performing the subject matter described in the instant disclosure. In some implementations, one or more components of the computer (902) may be configured to operate within environments, including cloud-computing-based, local, global, or other environment (or a combination of environments).

At a high level, the computer (902) is an electronic computing device operable to receive, transmit, process, store, or manage data and information associated with the described subject matter. According to some implementations, the computer (902) may also include or be communicably coupled with an application server, e-mail server, web server, caching server, streaming data server, business intelligence (BI) server, or other server (or a combination of servers).

The computer (902) may receive requests over network (930) from a client application (for example, executing on another computer (902) and responding to the received requests by processing the said requests in an appropriate software application. In addition, requests may also be sent to the computer (902) from internal users (for example, from a command console or by other appropriate access method), external or third-parties, other automated applications, as well as any other appropriate entities, individuals, systems, or computers.

Each of the components of the computer (902) may communicate using a system bus (903). In some implementations, any or all of the components of the computer (902), both hardware or software (or a combination of hardware and software), may interface with each other or the interface (904) (or a combination of both) over the system bus (903) using an application programming interface (API) (912) or a service layer (913) (or a combination of the API (912) and service layer (913). The API (912) may include specifications for routines, data structures, and object classes. The API (912) may be either computer-language independent or dependent and refer to a complete interface, a single function, or even a set of APIs. The service layer (913) provides software services to the computer (902) or other components (whether or not illustrated) that are communicably coupled to the computer (902). The functionality of the computer (902) may be accessible for all service consumers using this service layer. Software services, such as those provided by the service layer (913), provide reusable, defined business functionalities through a defined interface. For example, the interface may be software written in JAVA, C++, or other suitable language providing data in extensible markup language (XML) format or another suitable format. While illustrated as an integrated component of the computer (902), alternative implementations may illustrate the API (912) or the service layer (913) as stand-alone components in relation to other components of the computer (902) or other components (whether or not illustrated) that are communicably coupled to the computer (902). Moreover, any or all parts of the API (912) or the service layer (913) may be implemented as child or sub-modules of another software module, enterprise application, or hardware module without departing from the scope of this disclosure.

The computer (902) includes an interface (904). Although illustrated as a single interface (904) in FIG. 9, two or more interfaces (904) may be used according to particular needs, desires, or particular implementations of the computer (902). The interface (904) is used by the computer (902) for communicating with other systems in a distributed environment that are connected to the network (930). Generally, the interface (904) includes logic encoded in software or hardware (or a combination of software and hardware) and operable to communicate with the network (930). More specifically, the interface (904) may include software supporting one or more communication protocols associated with communications such that the network (930) or interface's hardware is operable to communicate physical signals within and outside of the illustrated computer (902).

The computer (902) includes at least one computer processor (905). Although illustrated as a single computer processor (905) in FIG. 9, two or more processors may be used according to particular needs, desires, or particular implementations of the computer (902). Generally, the computer processor (905) executes instructions and manipulates data to perform the operations of the computer (902) and any algorithms, methods, functions, processes, flows, and procedures as described in the instant disclosure.

The computer (902) also includes a memory (906) that holds data for the computer (902) or other components (or a combination of both) that may be connected to the network (930). The memory may be a non-transitory computer readable medium. For example, memory (906) may be a database storing data consistent with this disclosure. Although illustrated as a single memory (906) in FIG. 9, two or more memories may be used according to particular needs, desires, or particular implementations of the computer (902) and the described functionality. While memory (906) is illustrated as an integral component of the computer (902), in alternative implementations, memory (906) may be external to the computer (902).

The application (907) is an algorithmic software engine providing functionality according to particular needs, desires, or particular implementations of the computer (902), particularly with respect to functionality described in this disclosure. For example, application (907) may serve as one or more components, modules, applications, etc. Further, although illustrated as a single application (907), the application (907) may be implemented as multiple applications (907) on the computer (902). In addition, although illustrated as integral to the computer (902), in alternative implementations, the application (907) may be external to the computer (902).

There may be any number of computers (902) associated with, or external to, a computer system containing computer (902), wherein each computer (902) communicates over network (930). Further, the term “client,” “user,” and other appropriate terminology may be used interchangeably as appropriate without departing from the scope of this disclosure. Moreover, this disclosure contemplates that many users may use one computer (902), or that one user may use multiple computers (902).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method, comprising:
obtaining a first input data indicative of properties of a first liquid sample, the first input data comprising turbidity data and total suspended solids data for the first liquid sample, wherein the first liquid sample is acquired from a liquid source;
determining, using a computer processor and a machine learning model, a first predicted particle-size distribution of the first liquid sample based on the first input data, wherein particle-size distribution is controlled, at least in part, by a set of dosage parameters configurable by a water quality system;
determining, with an optimizer applied to the machine learning model, an optimal set of dosage parameters based on the first predicted particle-size distribution; and
adjusting the set of dosage parameters of the water quality system to the optimal set of dosage parameters.

2. The method of claim 1, further comprising:
determining a dosage rate for a chemical based on the first predicted particle-size distribution, and injecting the chemical into the liquid source at the dosage rate, wherein the dosage rate is comprised by the optimal set of dosage parameters.

3. The method of claim 1, wherein the machine learning model is a support vector machine.

4. The method of claim 1, further comprising processing, using the computer processor, the input data, wherein the processing includes normalizing the data.

5. The method of claim 1, wherein determining, with the optimizer, the optimal set of dosage parameters comprises maximizing a particle aggregation.

6. The method of claim 5,
wherein maximizing the particle aggregation comprises increasing a median of the first predicted particle-size distribution.

7. The method of claim 1, further comprising:
obtaining a second input data indicative of properties of a second liquid sample, the second input data comprising turbidity data and total suspended solids data for the second liquid sample, wherein the second liquid sample is collected after adjusting the set of dosage parameters;
determining, using the computer processor and the machine learning model, a second predicted particle-size distribution of the second liquid sample based on the second input data; and
validating the optimal set of dosage parameters with a determination that a particle aggregation of the second liquid sample is increased relative to a particle aggregation of the first liquid sample based on the first and second predicted particle-size distributions.

8. The method of claim 1, further comprising:
measuring the particle-size distribution of a second liquid sample, wherein the second liquid sample is collected after adjusting the set of dosage parameters; and
validating the optimal set of dosage parameters with a determination that a particle aggregation of the second liquid sample is increased relative to a particle aggregation of the first liquid sample, the particle aggregation of the first liquid sample determined using the first predicted particle-size distribution.

9. The method of claim 1, further comprising:
determining, using the computer processor and the machine learning model, a quality assessment metric based on the first particle-size distribution of the first liquid sample; and
generating one or more alerts regarding liquid quality based, at least in part, on the quality assessment metric.

10. The method of claim 9,
wherein the quality assessment metric comprises a liquid quality level,
wherein the one or more alerts are generated based on a determination that the liquid quality level is lower than an acceptable liquid quality level.

11. The method of claim 1, further comprising:
determining, using the computer processor and the machine learning model, a trend analysis data based, at least in part, on the first predicted particle-size distribution; and
generating, using the computer processor and the machine learning model, a liquid quality report based, at least in part, on the first predicted particle-size distribution.

12. The method of claim 1,
wherein the input data further comprises environmental parameter data comprising a temperature and a pH level of the liquid source.

13. A water quality system, comprising:
a plurality of sensors configured to measure property data of, at least, a liquid sample acquired from a liquid source; and
a control system configured to adjust a set of dosage parameters of one or more chemicals used by the water quality system, the control system in communication with the plurality of sensors comprising a processor and a memory, the memory storing instructions that, when executed by the processor, cause the processor to:
obtain input data for a first liquid sample from the plurality of sensors, the input data comprising turbidity data and total suspended solids data for the first liquid sample, wherein the first liquid sample is acquired from the liquid source;
determine, using a machine learning model, a first predicted particle-size distribution of the first liquid sample based on the input data, wherein particle-size distribution is controlled, at least in part, by the set of dosage parameters;
determine, with an optimizer applied to the machine learning model, an optimal set of dosage parameters based on the first predicted particle-size distribution; and
adjust the set of dosage parameters to the optimal set of dosage parameters.

14. The system of claim 13, further comprising:
determining a dosage rate for a chemical based on the first predicted particle-size distribution, and injecting the chemical into the liquid source at the dosage rate, wherein the dosage rate is comprised by the optimal set of dosage parameters.

15. The system of claim 13, wherein determining, with the optimizer, the optimal set of dosage parameters comprises maximizing a particle aggregation.

16. The system of claim 15,
wherein maximizing the particle aggregation comprises increasing a median of the first predicted particle-size distribution.

17. The system of claim 13, further comprising:
determining, using the machine learning model, a quality assessment metric based on the first particle-size distribution; and
generating one or more alerts regarding liquid quality based, at least in part, on the quality assessment metric.

18. The system of claim 13,
wherein the machine learning model is a support vector machine.

19. A non-transitory computer-readable medium storing instructions that, when executed by a processor, cause the processor to perform a method comprising:
obtaining input data indicative of properties of a first liquid sample, the input data comprising turbidity data and total suspended solids data for the first liquid sample, wherein the first liquid sample is acquired from a liquid source;
determining, using a machine learning model, a first predicted particle-size distribution of the first liquid sample based on the input data, wherein particle-size distribution is controlled, at least in part, by a set of dosage parameters configurable by a water quality system;
determining, with an optimizer applied to the machine learning model, an optimal set of dosage parameters based on the first predicted particle-size distribution; and
adjusting the set of dosage parameters of the water quality system to the optimal set of dosage parameters.

20. The non-transitory computer-readable medium of claim 19, wherein determining, with the optimizer, the optimal set of dosage parameters comprises maximizing a particle aggregation of the liquid source from which the first liquid sample was obtained.
